(19) United States
(12) Patent Application Publication
(10) Pub. No.: US 2025/0369017 A1
(43) Pub. Date: Dec. 4, 2025

(54) IMPROVED RECOMBINANT ADENO-ASSOCIATED VIRUS PRODUCTION

(71) Applicant: BRAMMER BIO, LLC, Cambridge, MA (US)

(72) Inventors: Hetal Brahmbhatt, St Augustine, FL (US); Jessica Tate, East Concord, NY (US); Ratna Karan, Gainesville, FL (US); Maria Colombo, Boncelles (BE); Richard Snyder, Gainesville, FL (US)

(21) Appl. No.: 18/877,139

(22) PCT Filed: Jun. 20, 2023
(86) PCT No.: PCT/US2023/025778

§ 371 Date: Dec. 19, 2024

Related U.S. Application Data
(60) Provisional application No. 63/353,793, filed on Jun. 20, 2022.

(51) Int. Cl. C12N 15/864; C12N 5/10; C12N 15/113

(52) U.S. Cl. CPC C12N 15/8645; C12N 5/10; C12N 15/1131; C12N 2310/14; C12N 2310/531; C12N 2510/00; C12N 2750/14143; C12N 2750/14152; C12N 2800/107

(57) ABSTRACT

Application of adeno-associated virus (AAV) vectors in large animal studies and clinical trials often requires high-titer and high-potency vectors manufactured at a scale that can support large doses and/or large populations. A number of currently used vector production methods, based on either transient transfection or helper virus infection of cell lines, have their advantages and limitations. Here we report novel methods and compositions for high-titer AAV production with several key improvements and advantages: (1) a one-step cloning of therapeutic AAV vector cassette into the serotype-specific packaging plasmid; (2) a single plasmid transfection and selection for stable AAV vector producer cell lines.

### RELATED APPLICATIONS

This application claims the benefit of International Patent Application PCT/US2023/025778, filed Jun. 20, 2023 which claims benefit of U.S. Provisional Patent Application Ser. No. 63/353,793, filed Jun. 20, 2022, the disclosure of which is hereby incorporated by reference in its entirety.

### BACKGROUND

Delivering a therapeutic nucleic acid to cells is an important biomedicine technique, both as a tool in basic research and a means for cell and gene therapy. Recombinant Adeno-associated viruses (rAAVs), engineered forms of Adeno-associated viruses (AAVs), have been used for in vitro and in vivo gene delivery due to their high transduction efficiency, safety, and extended stable gene expression. This constellation of favorable features has resulted in approved rAAV based gene therapies; Glybera for lipoprotein lipase deficiency, Luxturna for Leber's congenital amaurosis, and Zolgensma, for spinal muscular atrophy type 1.

Common to all natural and rAAVs are their small, nonenveloped icosahedral capsids, ˜260 Å in diameter, that contain a linear single-stranded DNA genome. Natural AAVs have a genome size of approximately 4.7 kb. Both ends of the natural AAV genome contain inverted terminal repeats (ITRs); 145 nucleotides (nt) that form T-shaped hairpin secondary structures that are important for genome replication and packaging. Between these ITRs, two open reading frames (ORFs) encode a series of replication (Rep) and capsid (Cap) proteins, and other proteins (e.g., assembly-activating protein (AAP)).

ITRs contain all the cis-acting elements involved in replicating and packaging of the AAV genome, thus rAAV vector design can rely on removal of the viral genes. In this design, rAAV particles are generated by introducing into cells a plasmid (AAV cis-plasmid) containing a cloned rAAV genome composed of foreign DNA flanked by the 145 nucleotide-long ITRs and a separate construct expressing in trans the AAV viral genes (rep, cap, AAP, etc). The helper factors are provided either by adenovirus infection of the cell or by introduction of a third plasmid that provides these adenovirus helper factors.

As one can surmise, successfully delivering three plasmids to one cell is a relatively inefficient process. For larger-scale manufacturing efforts, transient delivery of plasmid requires excess quantities of DNA, adding to the overall cost of production and purification. Moreover, transient delivery of rep/cap genes in the presence of helper genes can also contribute to product heterogeneity, including rAAV capsids that lack a genome. These “empty capsids” represent a significant proportion of virus produced in a transient transfection process. Thus, there remains a critical need in the art for improved methods, systems and compositions permitting the efficient production of rAAV for use as vectors for somatic gene therapy.

### BRIEF SUMMARY

rAAV are commonly used as a powerful tool for in vivo gene transfer. rAAV has been successfully used to establish efficient and long-term gene transfer in a variety of targeted tissues as well as systemically. Although the applications of rAAV offer great potential for curing many genetic diseases, current rAAV production methods still have room for improvement to meet clinical demands, especially those requiring large doses of high-quality vectors and disease indications that affect large populations. Accordingly, this prompted us to develop unique methods and compositions to meet these demands.

Disclosed herein is a system for producing a recombinant adeno-associated virus (AAV) encompassing a stably transfected mammalian cell line and a helper nucleic acid. The stably transfected cell line possesses an exogenous DNA sequence composed of an AAV rep gene, and AAV cap gene, a gene of interest, and an RNAi sequence that targets the AAV rep gene. The helper nucleic acid possesses a sequence that increases AAV rep gene expression. In some embodiments, the exogenous DNA sequence further encompasses a second RNAi that targets the helper nucleic acid.

In some embodiments, disclosed herein is an isolated nucleic acid encompassing an adeno-associated virus (AAV) rep gene, an AAV cap gene, a RNAi sequence, the RNAi sequence targeting an AAV gene transcript, a selectable marker capable of both prokaryotic and eukaryotic selection, and a gene of interest, such as a therapeutic nucleic acid (could also be a marker gene) flanked on each end by an AAV inverted terminal repeat (ITR). In other embodiments, the isolated nucleic acid further encompasses a second RNAi sequence, the second RNAi sequence targeting the helper nucleic acid.

Still further disclosed herein is a method of producing a stably transfected cell line, the method encompassing transfecting a host cell line with an isolated nucleic acid, the isolated nucleic acid encompassing an AAV rep gene, an AAV cap gene, an interfering RNA (RNAi) sequence, the RNAi sequence targeting an AAV gene transcript, a selectable marker capable of both prokaryotic and eukaryotic selection, and a gene of interest flanked by on each end by an AAV inverted terminal repeat. In some embodiments, the method further encompasses a second RNAi sequence, the second RNAi sequence targeting a helper nucleic acid.

Also disclosed herein, is a method for producing a rAAV drug substance, the method encompassing introducing a helper nucleic acid to a stably transfected mammalian cell line and thereby producing an rAAV drug substance.

### DESCRIPTION OF THE DRAWINGS

FIG. 1. An exemplary plasmid map used in establishing a cell line for producing a rAAV drug substance. Starting approximately from the 12 o'clock position on the plasmid map and continuing clockwise, the plasmid includes a promoter driving transcription of AAV rep and cap, a promoter driving transcription of an shRNA directed to the rep transcript, an ITR sequence, a promoter driving transcription of a gene of interest (GOI), in this instance the promoter being from the cytomegalovirus (CMV), another ITR, thereby causing the GOI to be flanked by ITR sequences, next a promoter driving transcription of an shRNA directed to a adenovirus transcript, a further promoter driving transcription of selectable marker, in this instance, a promoter that is capable of initiating transcription in prokaryotes and eukaryotes, and a marker capable of selecting for prokaryotes and eukaryotes harboring the plasmid.

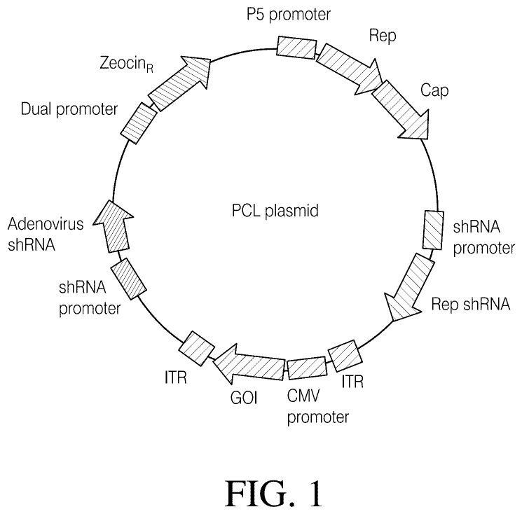

FIG. 2. A further exemplary plasmid map used in establishing a cell line for producing a rAAV drug substance. Starting from approximately the 12 o'clock position on the plasmid map and continuing clockwise, the plasmid includes an insulator sequence, a promoter for driving transcription of AAV rep and cap, in this instance, the rep and cap sequences are derived from AAV2, but they could be derived from any AAV serotype, and even mixed, for instance AAV2 rep and AAV8 cap, an insulator sequence, a promoter driving transcription of an shRNA directed to an adenovirus transcript, in this instance tripartite leader (TPL), and insulator sequence, an ITR sequence, a promoter for driving transcription of a GOI, in this instance the promoter being from the cytomegalovirus (CMV) and the GOI being a fusion transcript between human β-globulin and Emerald Green Fluorescent Protein (emGFP), another ITR, thereby causing the GOI to be flanked by ITR sequences, an insulator sequence, a promoter driving transcription of an shRNA directed to the rep transcript, an insulator, and a selectable marker, the marker capable of selecting for prokaryotes and eukaryotes harboring the plasmid, in this instance the selectable marker is Zeocin™ selection antibiotic.

FIG. 3. A table depicting unique short hairpin RNA (shRNA) targets within the adenovirus tripartite leader (TPL) and AAV rep nucleic acid sequences and a BLAST search demonstrating an absence, denoted with an “X,” of off-target homology within the human genome, human Adenovirus type 5 off-target sequence, and AAV serotype 2 off-target sequences. Target homologous sequences are indicated with a V symbol. Targeted TPL and rep sequences lack homology to each other.

FIG. 4. Evaluation of various RNA pol III promoters, U6, H1, H1-Hy and 7SK, in driving shRNA mediated knockdown of gfp mRNA levels in transfected A549 cells. To test the promoters, A549 cells were transfected with one of four plasmids capable of transcribing green fluorescent protein (gfp) and an shRNA targeting gfp whose transcription was driven by one of four promoters. GFP transcription levels were then measured using reverse transcriptase polymerase chain reaction (RT-PCR). The figure shows that each tested promoter, U6, H1, H1-Hy and 7SK, drove sufficient transcription of an shRNA targeting gfp to reduce gfp mRNA levels relative to a control, “scramble.” X-axis, comparison of each promoter driving scramble and shRNA targeting gfp. Y-axis, relative mRNA levels of gfp.

FIG. 5. Adenovirus vector genome titer in the presence of A549 cells expressing control and various shRNAs targeting the adenovirus tripartite leader (TPL). The ability of different shRNA sequences targeting the adenovirus TPL sequence to reduce adenovirus genome titer was tested at various multiplicity of infection (MOI) of transfected A549 cells. At every MOI, shRNAs targeting the TPL reduced the concentration of detected adenovirus genomes. X-axis, multiplicity of infection. Y-axis, adenovirus genomes per/ml of culture media. Negative control-“scramble,” a randomly generated shRNA sequence without identity to TPL. TPL #1-4 represents shRNA sequences targeting TPL. IVa2 represents a shRNA sequence that targets the adenovirus intermediate gene IVa2.

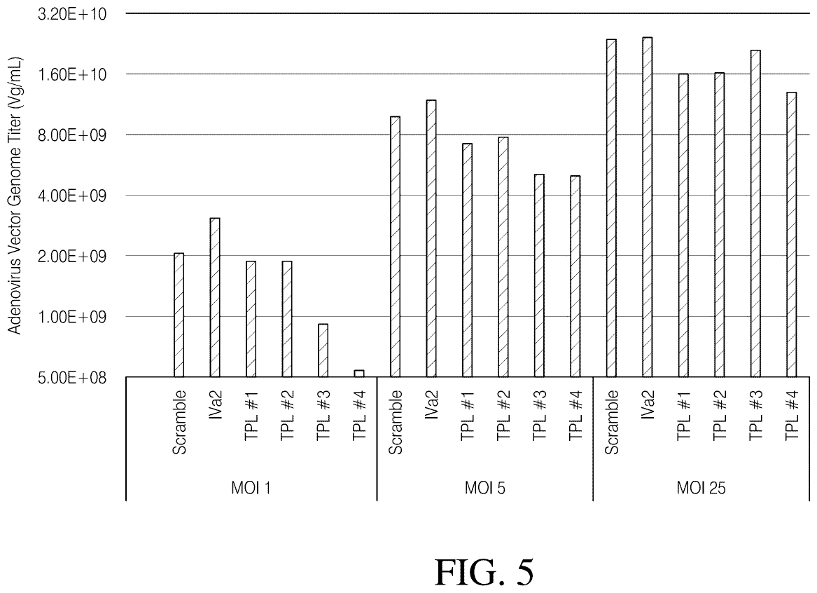

FIG. 6. rep mRNA levels in the presence of control shRNA, “scramble,” a sequence lacking identity to rep, or five shRNAs targeting rep (REP 1-5) in HEK293 cells. Various shRNA sequences were evaluated for the ability to reduce rep mRNA levels. rep transcription levels were normalized in each condition using endogenous 18S rRNA levels. X-axis, test shRNAs. Y-axis relative mRNA levels.

FIG. 7. A comparison of linearized and circular plasmid in transfected cell lines. Various cell lines, BHK21, HeLa, HeLa S and A549, were transfected with either linearized or circular plasmid harboring a zeocin antibiotic resistance cassette. The number of surviving colonies of each cell line, either transfected with linearized or circular plasmid, at two different zeocin concentrations, was compared. X-axis, tested conditions including cell line and zeocin concentrations. Y-axis, colony numbers (counts).

### DETAILED DESCRIPTION

“Recombinant adeno-associated virus” (rAAV) refers to a virus particle that functions as a nucleic acid delivery vehicle, carrying a therapeutic nucleic acid packaged within an AAV capsid. rAAV vector-based gene therapy has been adapted for use in more than 100 clinical trials. This is because of its excellent safety profile, ability to target a wide range of tissues, stable transgene expression, and significant clinical benefit. However, a major challenge is to produce at large-scale a high-titer, high-potency vector to achieve a better therapeutic effect.

Commonly applied rAAV production methods rely on either transient transfection of mammalian cells or infection of mammalian or insect cells. These strategies require the successful introduction of multiple, independent nucleic acid molecules to the same cell.

In transient transfection, two or even three plasmids need to be introduced to the same cell in a narrow time window. Similarly, infection-based strategies require co-infection of the same cell with two or three viruses. The need to introduce multiple nucleic acid molecules, in a short time, to the same cell, makes these methods labor intensive, difficult to scale and expensive.

Stably transfected producer cell lines are an alternate approach that overcomes the limitations of the transient transfection and infection-based methods. In this approach a cell line is generated that can pass copies of AAV viral genes and a therapeutic nucleic acid flanked by ITRs to their progeny by incorporation into the production cell's genome. Production of rAAV is induced in this approach by infecting this stable cell line with a helper virus, for instance adenovirus. To engineer such a stable producer cell line an exogenous nucleic acid, such as nucleic acid vector, is utilized.

### Nucleic Acid Vector

A nucleic acid vector refers to a vehicle that carries an exogenous DNA sequence into a target cell. A nucleic acid vector can be engineered to possess one or more selection gene(s) in order to select or to identify cells harboring the exogenous DNA sequence, one or more stabilizing elements to maintain the nucleic acid vector within the cell (for example, cer sequence) and/or one or more integrative elements (for example, LTR viral sequences, transposons and recombination sites) that facilitate the integration of nucleic acid vector into a host cell's genome. Examples of nucleic acid vectors include bacterial artificial chromosomes (BACs), yeast artificial chromosomes (YACs), P1-derived artificial chromosomes (PACs), cosmids, and recombinant DNA plasmids. In some embodiments, the nucleic acid vector is a recombinant plasmid.

Beyond a selection gene, stabilizing elements or integrative elements, a nucleic acid vector carries other exogenous genetic material into a target cell. In some embodiments, the exogenous DNA sequence carried, in part, by the nucleic acid vector includes one or more sequences that encode agents that repress the translation of an AAV Rep protein and one or more sequences that encode agents that repress the translation of a helper virus protein; for instance, repressing translation of adenovirus late proteins by targeting the adenovirus tripartite leader (TPL).

“AAV Rep protein” refers to one or more non-structural proteins that mediate AAV replication. Four AAV Rep proteins, Rep78, Rep68, Rep52, Rep40, result from the translation of alternatively spliced transcripts initiated from two promoters, p5 and p19. The p5 promoter produces a transcript encoding Rep78 and Rep68, while p19 produces a transcript encoding Rep52 and Rep40. In some embodiments, the nucleic acid vector encodes an agent that represses the translation of Rep78 and Rep68. In other embodiments, the nucleic acid vector encodes an agent that represses the translation of Rep52 and Rep40. In still other embodiments, the nucleic acid vector encodes an agent that represses the translation of Rep78, Rep68, Rep52 and Rep40.

The Rep proteins may be from any AAV, including but not limited to natural serotypes 1, 2, 3a, 3b, 4, 5, 6, 7, 8, 9, 10, 11, or 13 and any other AAV serotypes or variants known at the time of filing. Codon optimization/codon alterations can be applied/made to rep sequences. Such changes can alter translation of rep in cell specific ways, increasing or decreasing translation in a cell. In some embodiments, the nucleic acid vector encodes an agent that represses the translation of a naturally occurring rep. In other embodiments, the nucleic acid vector encodes an agent that represses the translation of a non-naturally occurring rep. In still other embodiments, the nucleic acid vector encodes an agent that represses the translation of a non-naturally occurring rep, the rep being one that has undergone codon optimization or other modification.

“AVV cap” refers to the proteins encoded by the cap gene existing in the AAV genome. The cap gene encodes three capsid proteins, VP1, VP2 and VP3, all of which form the capsid. Many naturally occurring and engineered capsid proteins are known as of the time of this application's filing. Some variants are called serotypes, designated 1, 2, 3 (A and B), 4-11. In some embodiments, the exogenous DNA sequence provides an AAV cap gene encoding an AAV cap.

A “gene of interest” or “GOI” refers to a polynucleotide sequence, such as a gene, an open reading frame, or a coding sequence for a protein. The encoded protein is used to moderate or remedy a disease or indication. When flanked by AAV inverted terminal repeats (ITRs), the gene of interest can be packaged within the AAV cap.

In addition to one or more sequences that encode agents that repress the translation of an AAV Rep protein, the exogenous DNA sequence, in certain circumstances, further encodes one or more agents that repress the translation of a helper nucleic acid. A “helper nucleic acid” is a polynucleotide that increases translation of rep.

“Helper virus” refers to a virus used to induce production of rAAV. The helper virus infects cells harboring an exogenous DNA sequence, such as a nucleic acid vector, and provides the necessary factors needed for rAAV replication and production. Helper viruses used to produce rAAV include adenovirus, herpesviruses, Epstein-Barr virus, cytomegalovirus, pox virus, bocavirus and vaccinia virus. In some embodiments, the exogenous DNA sequence encodes an agent that represses the translation of one or more adenovirus, herpesviruses, Epstein-Barr virus, cytomegalovirus, pox virus, bocavirus or vaccinia virus proteins. In some embodiments, the exogenous DNA sequence encodes an agent that represses the translation of one or more adenovirus proteins. In other embodiments, the exogenous DNA sequence encodes an agent that represses the translation of one or more adenovirus proteins but not adenovirus early region 1 (E1). In some embodiments, the exogenous DNA sequence encodes an agent that represses the translation of an adenovirus intermediate transcription unit. The adenovirus intermediate transcription unit includes IX, IVa2, L4 intermediate. In still other embodiments, the exogenous DNA sequence encodes an agent that represses the proteins translated from the major late transcription unit (MLTU). The MLTU encodes multiple proteins from five regions, L1 to L5, through differential splicing and polyadenylation. In other embodiments, the exogenous DNA sequence encodes an agent that represses the proteins translated from the major late transcription unit solely. In still other embodiments, the exogenous DNA sequence encodes an agent that represses L1, L2, L3, L4 or L5. In some embodiments, the exogenous DNA sequence encodes an agent that represses L1 or L4. In some embodiments, the exogenous DNA sequence encodes an agent that represses L4. In each instance, the percentage repression relative to a control without repression can be 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 98%, 99% or 100%. In some embodiments, the agent is a RNAi (see below). In other embodiments, the agent is a shRNA (see below).

Multiple mRNAs are produced from the MLTU all of which contain the tripartite leader (TPL) sequence. In some embodiments, the exogenous DNA sequence encodes an agent that represses the translation of proteins encoded by an mRNA containing the TPL sequence. In other embodiments, the exogenous DNA sequence encodes an agent that represses the translation of proteins encoded by an mRNA containing the TPL sequence solely. In each instance, the percentage repression relative to a control without repression can be 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 98%, 99% or 100%.

In some embodiments, a system is provided for producing a recombinant adeno-associated virus, the system encompassing a stably transfected mammalian cell line and a helper virus, wherein the stably transfected mammalian cell line possesses an exogenous DNA sequence, the exogenous DNA sequence having a AAV rep gene, an AAV cap gene, a gene of interest and an RNAi sequence. In other embodiments, the system includes a second RNAi sequence.

### RNAi

RNAi molecules can modulate expression of one or more gene(s) in a producer cell line. Typically, RNAis are used to reduce translation of one or more proteins.

RNAi molecules can be designed to antagonize translation by sequence homology-based targeting of the corresponding RNA sequence. Such RNAis will typically be small interfering RNAs (siRNAs), small hairpin RNAs (shRNAs), double strand RNA (dsRNA) or micro-RNAs (miRNAs). The sequence of such RNAis will encompass a portion that corresponds with that of a portion of the mRNA encoding the one or more proteins whose translation is to be repressed. This portion will usually be 100% complementary to the target portion within the mRNA transcribed from the one or more gene(s), but lower levels of complementarity (for example, 50% or more, 55% or more, 60% or more, 65% or more, 70% or more, 75% or more, 80% or more, 85% or more, 90% or more or 95% or more) may also be used. Typically, the percent (%) complementarity is determined over a length of contiguous nucleic acid residues. A RNAi molecule can, for example, have at least 50%, 55%, 60%, 65%, 70%, 75%, 80%, 85%, 90%, 95%, 98%, 99% or 100% complementarity to the target portion within the mRNA transcribed from the one or more genes measured over at least 10, at least 20, at least 30, at least 40, at least 50, at least 60, at least 70, at least 80, at least 90, or more nucleic acid residues up to length of the mRNA transcribed from the one or more gene(s) of interest, including those of rep, MLTU and/or TPL. In some instances, a dsRNA molecule can, for example, have at least 50%, 55%, 60%, 65%, 70%, 75%, 80%, 85%, 90%, 95%, 98%, 99% or 100% complementarity to the target portion within the mRNA transcribed from the one or more genes measured over at least 10, at least 20, at least 30, at least 40, at least 50, at least 60, at least 70, at least 80, at least 90, or more nucleic acid residues up to length of the mRNA transcribed from the one or more gene(s) of interest, including those of rep, MLTU and/or TPL but not adenovirus E1.

In some embodiments, the RNAi is a shRNA. ShRNA can be delivered to a host cell line by any appropriate means. Suitable techniques are known in the art and include the use of plasmid, viral and bacterial vectors to deliver the shRNA to the producer cell line. The shRNA in some embodiments is delivered using a plasmid delivery system.

Generally, once the shRNA has been delivered to a cell, it is then transcribed in the nucleus and processed. The resulting pre-shRNA is exported from the nucleus and then processed by dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both cases, the shRNA leads to target gene silencing.

### Selection Genes

A nucleic acid vector can include a selection gene or genes to select or to identify transfected cells, for example by complementation of a cell auxotrophy or by antibiotic resistance. Certain antibiotics are only useful in selecting for microbial cells while other antibiotics can be used to select for both prokaryotic and eukaryotic cells possessing an antibiotic resistance gene. Blasticidin, hygromycin B, mycophenolic acid, puromycin and Zeocin are examples of antibiotics that can be used to select for prokaryotic and eukaryotic cells harboring the appropriate antibiotic resistance gene.

Accordingly, in some embodiments the nucleic acid vector is a plasmid, the plasmid encompassing a selection gene. In some embodiments, the selection gene complements a cell auxotrophy. In other embodiments, the selection gene confers antibiotic resistance. In still other embodiments, the selection gene confers antibiotic resistance in both prokaryotic and eukaryotic cells.

Nucleic acid vectors can exist as an extrachromosomal nucleic acid capable of autonomous replication. BACs, YACs, PACs, cosmids and plasmids possess a non-mammalian origin of replication. A non-mammalian origin of replication is a nucleic acid sequence that enables the nucleic acid vectors to stably replicate and segregate alongside endogenous chromosome(s) in a suitable host cell (a microbial cell, such as a bacterial or yeast cell). Examples of non-mammalian origins of replication include bacterial origins of replication, such as oriC, oriV, oriS, or yeast origins of replications, also referred to as Autonomously Replicating sequences (ARS elements).

The nucleic acid vector can be introduced into a host cell as a linear nucleic acid or as a closed circle, such as a plasmid. In some embodiments, the nucleic acid vector is introduced into a host cell as a linear nucleic acid. In other embodiments, the nucleic acid vector is introduced into a host cell as a circular nucleic acid.

In some embodiments, the nucleic acid vector is less than 25, 20, 19, 18, 17, 16, 15, 14, 13, 12, 11, 10, 9, 8, 7, 6 or 5kb in length.

The nucleic acid vector can integrate into the host cell genome and so become incapable of autonomous replication. The integration of the nucleic acid vector into the host cell genome can occur randomly or be targeted. Integration can occur at one or more locations within the host cell genome. In some embodiments, the nucleic acid vector is integrated into the host cell genome at an untargeted, random location. In other embodiments, the nucleic acid vector is integrated into the host cell genome at a predetermined, targeted location. In some embodiments, the nucleic acid vector is integrated into the host cell genome at a predetermined, targeted location, the targeted location being on the long arm of chromosome 19, termed AAVI site. In some embodiments, the nucleic acid vector is integrated into the host cell genome at a predetermined, targeted location, the targeted location being engineered within the genome of the host cell, for instance a safe harbor site.

Integration into a Safe Harbor Locus

The nucleic acid vector can be inserted into a specific safe harbor location in the genome. Several safe harbor loci have been described, including AAVS1, CCR5, and Rosa26. Other safe harbor loci have been identified, for instance, as described in WO/2019/169232 entitled “Identifying and characterizing genomic safe harbors (gsh) in humans and murine genomes, and viral and non-viral vector compositions for targeted integration at an identified gsh loci.” In alternative embodiments, the disclosure herein also relates to nucleic acid vector compositions comprising a-safe harbor (SH)-5′ homology arm, and a 3′SH homology arm flanking a nucleic acid comprising a restriction cloning site, where the vector can be used to integrate the flanked nucleic acid into the genome at an SH by homologous recombination. In all aspects as disclosed herein, the nucleic acid vector compositions can be a plasmid, cosmid, or artificial chromosome (e.g., BAC), minicircle nucleic acid, doggy bone DNA, or recombinant viral vector (e.g., rAd, AAV, rHSV, BEV or variants thereof).

Accordingly, one aspect of the technology described herein relates to a nucleic acid vector composition comprising: (a) a genomic safe harbor (GSH) 5′ homology arm (also referred to herein as “5′ GSH-specific homology arm” or “5′ GSH-HA”), (b) a nucleic acid sequence comprising a restriction cloning site, and (c) a GSH 3′ homology arm (also referred to herein as “3′ GSH-specific homology arm” or “3′ GSH-HA”), where the 5′ homology arm and the 3′ homology arm bind to a target site located in a genomic safe harbor locus, and wherein the 5′ and 3′ homology arms allow insertion (of the nucleic acid located between the homology arms) by homologous recombination into a loci located within the genomic safe. In some embodiments, a nucleic acid vector composition for integration of a nucleic acid of interest into a GSH loci comprises a nucleic acid of interest and/or an expressible transgene cassette (e.g., a sequence that encodes genes, a gene editing molecule described herein, or a reporter protein) and can include one or more gene editing molecules.

In some embodiments, a nucleic acid vector composition for integration of a nucleic acid of interest into a GSH loci as described herein comprises in this order: a) a 5′ GSH-specific homology arm, c) a restriction cloning site, and d) a 3′ GSH-specific homology arm. In some embodiments, the 3′ and 5′ homology arms complementary base pair with regions of the GSH. In some embodiments, 3′ and 5′ homology arms flank a target site of integration, e.g., target insertion loci in the GSH. In some embodiments, the 3′ homology arm complementary base pairs with a nucleic acid region 3′ (i.e., upstream) of a target site of integration or target insertion loci of the GSH, and 5′homology arm complementary base pairs with a nucleic acid region 5′ (i.e., downstream) of a target site of integration or target insertion loci of the GSH. In some embodiments, the 5′ and 3′ homology arms are complementary to, e.g., at least 60%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99%, or at least 99.5% complementary to portions of the GSH.

To increase the likelihood of integration at a precise location, the 5′ and 3′ homology arms may include enough nucleic acids, such as 50 to 5,000 base pairs, or 100 to 5,000 base pairs, or 500 to 5,000 base pairs, which have a high degree of sequence identity or homology to the corresponding target sequence to enhance the probability of homologous recombination. The 5′ and 3′ homology arms may be any sequence that is homologous with the GSH target sequence in the genome of the host cell. That is, the 5′ and 3′ homology arms are complementary to portions of the GSH target sequence identified herein. Furthermore, the 5′ and 3′ homology arms may be non-encoding or encoding nucleotide sequences. In some embodiments, the homology between the 5′ homology arm and the corresponding sequence on the chromosome is at least any of 80%, 85%, 90%, 95%, 97%, 98%, 99%, or 100%. In embodiments, the homology between the 3′ homology arm and the corresponding sequence on the chromosome is at least any of 80%, 85%, 90%, 95%, 97%, 98%, 99%, or 100%. In embodiments, the 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least 1, 2, 5, 10, 15, 20, 25, 30, 50, 100, 200, 300, 400, or 500 bp away from the integration or DNA cleavage site, or partially or completely overlapping with the DNA cleavage site. In embodiments, the 3′ homology arm of the nucleotide sequence is proximal to the ITR.

In some embodiments, the 5′ and/or 3′ homology arm can be any length, e.g., between 30-2000bp. In some embodiments, the 5′ and/or 3′ homology arms are between 200-350bp long. Details regarding length of homology arms and recombination frequency is reported by Zhang et al. “Efficient precise knockin with a double cut HDR donor after CRISPR/Cas9-mediated double-stranded DNA cleavage.” Genome biology 18.1 (2017): 35, which is incorporated herein in its entity by reference.

In some embodiments, the GSH 5′ homology arm and the GSH 3′ homology arm bind to target sites that are spatially distinct nucleic acid sequences in the genomic safe harbor identified according to the methods as disclosed herein. In some embodiments, a nucleic acid vector composition as described herein for integration of a nucleic acid of interest into a GSH locus comprises a 5′ GSH-specific homology arm and the GSH 3′ GSH-specific homology arm that are at least 65% complementary to a target sequence in the genomic safe harbor locus identified according to the methods disclosed herein. In some embodiments, a nucleic acid vector composition as described herein for integration of a nucleic acid of interest into a GSH loci as disclosed encompasses a 5′ GSH-specific homology arm and the 3′ GSH-specific homology arm that bind to a target site located in the PAX5 genomic safe harbor sequence. In one embodiment the nucleic acid vector composition as described herein for integration of a nucleic acid of interest into a GSH locus does not contain any prokaryotic DNA sequence elements, for example minicircle-DNA (mcDNA), but it is contemplated that some prokaryotic-sourced DNA, such as an antibiotic resistance gene, can be inserted as an exogenous sequence. In some embodiments, a nucleic acid vector composition as described herein for integration of a nucleic acid of interest into a GSH loci is a plasmid or a double-stranded DNA. In one aspect, a nucleic acid vector composition for integration of a nucleic acid of interest into a GSH loci as described herein includes or is obtained from a plasmid encoding in this order: a nucleotide sequence of interest (for example an expression cassette of an exogenous DNA, gene editing sequence, or donor sequence) positioned between a 5′ homology arm and a 3′ homology arm.

### Transcriptional Control Sequences

The expression of genes is controlled at the level of initiation of transcription by proteins that bind to specific regulatory sequences. These regulatory sequences include promoters. A “Promoter” is a DNA sequence recognized by the transcriptional machinery to initiate transcription of a downstream polynucleotide sequence. Promoters control the binding of RNA polymerase to DNA to initiate transcription. There are three types of RNA polymerases that transcribe different genes. RNA polymerase I transcribes genes encoding ribosomal RNA.

RNA polymerase II synthesizes most small nuclear RNAs. “RNA polymerase II promoter” or “RNA pol II promoter” or “polymerase II promoter” or “pol II promoter” is meant any invertebrate, vertebrate, or mammalian promoter, e.g., human, murine, porcine, bovine, primate, simian, etc. that, in its native context in a cell, associates or interacts with RNA polymerase II to transcribe its operably linked gene, or any variant thereof, natural or engineered, that will interact in a selected host cell with an RNA polymerase II to transcribe an operably linked nucleic acid sequence. RNA pol II promoters include any higher eukaryotic, including any vertebrate or mammalian, promoter containing any sequence variation or alteration, either natural or produced in the laboratory, which maintains or enhances but does not abolish the binding of RNA polymerase II to said promoter, and which is capable of transcribing a gene or nucleotide sequence, either natural or engineered, which is operably linked to said promoter sequence. Examples of RNA polymerase II promoters include CMV, SV40, bacteriophage T7 and SP6.

“RNA polymerase III promoter” or “RNA pol III promoter” or “polymerase III promoter” or “pol III promoter” is meant any invertebrate, vertebrate, or mammalian promoter, e.g., human, murine, porcine, bovine, primate, simian, etc. that, in its native context in a cell, associates or interacts with RNA polymerase III to transcribe its operably linked gene, or any variant thereof, natural or engineered, that will interact in a selected host cell with an RNA polymerase III to transcribe an operably linked nucleic acid sequence. By U6 promoter (e.g., human U6, murine U6), HI promoter, or 7SK promoter is meant any invertebrate, vertebrate, or mammalian promoter or polymorphic variant or mutant found in nature to interact with RNA polymerase III to transcribe its cognate RNA product, i.e., U6 RNA, H1 RNA, or 7SK RNA, respectively. Preferred in some applications are the Type III RNA pol III promoters including U6, H1, and 7SK which exist in the 5′ flanking region, include TATA boxes, and lack internal promoter sequences. Internal promoters occur for the pol III 5S rRNA, IRNA or VA RNA genes. The 7SK RNA pol III gene contains a weak internal promoter and a sequence in the 5′ flanking region of the gene necessary for transcription. RNA pol III promoters include any higher eukaryotic, including any vertebrate or mammalian, promoter containing any sequence variation or alteration, either natural or produced in the laboratory, which maintains or enhances but does not abolish the binding of RNA polymerase III to said promoter, and which is capable of transcribing a gene or nucleotide sequence, either natural or engineered, which is operably linked to said promoter sequence.

In some embodiments, the RNAi promoter is an RNA pol II promoter. In other embodiments, the RNAi promoter is an RNA pol III promoter. In still other embodiments, the second RNAi promoter is an RNA pol II promoter of an RNA pol III promoter.

An RNA transcript typically includes the coding sequence of a gene and a poly A tail. “Poly A tail” refers to a chain of adenine nucleotides added to the end of an RNA transcript or is encoded by the DNA template. A poly A tail is typically 5-300 nucleotides in length.

In some embodiments, an exogenous nucleic acid is provided, the exogenous nucleic acid providing an AAV rep gene, an AAV cap gene, an RNAi sequence that targets AAV rep gene expression, a selectable marker and a gene of interest flanked by two AAV inverted terminal repeats, one ITR on each end of the gene of interest. In other embodiments the exogenous nucleic acid includes a second RNAi sequence, the second RNAi sequence targeting a helper virus nucleic acid or helper nucleic acid.

### Insulator Sequences

Eukaryotic genomes are organized into domains containing individual genes and gene clusters that have distinct patterns of expression both during development and in differentiated cells. These genomes contain regulatory elements such as enhancers that can activate target genes in cis over considerable distances. “Insulator” is the name given to a class of DNA sequence elements that possess a common ability to protect genes from inappropriate signals emanating from their surrounding environment, for instance enhancers. In some instances, the nucleic acid vector includes one or more insulator sequences.

### Producer Cell Line

A “Producer Cell Line” refers to a cell line capable of replicating and packaging an rAAV vector. Any appropriate producer cell line may be modified and used according to the present invention. A producer cell line of the invention is a eukaryotic cell line, and typically a mammalian cell line. The AAV vectors produced by the present invention are usually intended for therapy in humans. Therefore, preferably a producer cell line of the invention is a human cell line. A producer cell line of the invention may be selected from NIH3T3, HT1080, A549, HeLa, BHK21, and HEK293 cell lines. A producer cell line of the invention may be in an adherent or suspension form.

A producer cell line is used to form virus particles capable of infecting a target cell. Viral vectors used in gene therapy are generated by a producer cell line that packages a therapeutic nucleic acid into a viral particle. The vectors typically contain the minimal viral sequences required for packaging and subsequent integration into a target, other viral sequences being replaced by an expression cassette encoding the therapeutic nucleic acid to be expressed. The missing viral functions are supplied in trans.

In a preferred embodiment, the producer cell line is the human embryonic kidney (HEK) 293 cell line. The HEK293 cell line expresses the adenovirus early region.

Other AAV serotypes, including AAV1, AAV3, AAV4, AAV5, AAV6, AAV8, AAV8.2, AAV9, AAV rh10, and pseudotyped AAV such as AAV2/5, AAV2/6 and AAV2/8 and engineered capsids.

A stably transfected mammalian cell line, as used herein, is a producer cell line that has been transfected with a nucleic acid to produce cells with one or more genes inserted into their genome. This can be accomplished, in some circumstances, by linking a desired gene or genes to a selectable marker.

### Therapeutic Applications

The AAV vectors of the invention may be used as gene therapy vectors in vivo or used to modify cells ex vivo for cell-based therapies. Gene transfer involves the delivery of new genetic information into cells. Genetic information may be transiently or stably inserted into cells. The AAV vectors of the present invention are used for gene transfer. Thus, the AAV vectors of the present invention may be capable of non-site-specific or site-specific integration into a mammalian chromosome without substantial cytotoxicity, and which direct host cell-specific expression of a therapeutic polynucleotide. Preferably the AAV vectors of the invention are used in gene therapy in mammals, more preferably humans.

The AAV vectors of the invention may comprise any polynucleotide which can be used to treat or prevent a disease or condition. The therapeutic polynucleotide may be DNA or RNA. Typically, the therapeutic polynucleotide is DNA. The therapeutic polynucleotide is preferably a biologically functional nucleic acid which targets (replaces or modifies) a non-functional and/or mutated gene in the individual to be treated. In preferred embodiments, when the individual to be treated is human, the therapeutic polynucleotide is also human.

The therapeutic polynucleotide may encode a biologically functional protein, i.e., a polypeptide or protein which affects the cellular mechanism of a cell in which the biologically functional protein is expressed. For example, the biologically functional protein can be a protein which is essential for normal growth of the cell or for maintaining the health of an individual. The biologically functional protein can also be a protein which improves the health of an individual by either supplying a missing protein, by providing increased quantities of a protein which is under-produced in the individual or by providing a protein which inhibits or counteracts an undesired molecule which may be present in the individual. The biologically functional protein can also be a protein which is a useful protein for investigative studies for developing new gene therapies or for studying cellular mechanisms.

The biologically functional protein can be a protein which is essential for normal growth or repair of the body. The biologically functional protein may also be one which is useful in fighting diseases such as cancer. The biologically functional protein may also be a selectable marker for antibiotic resistance such as a selectable marker for neomycin resistance in eukaryotes. Other types of selectable markers may also be used. The therapeutic polynucleotide encoding these proteins can be provided by any of a variety of methods, such as routine cloning procedures, excision from a vector containing the gene of interest, or chemical or enzymatic synthesis based on published sequence information. In many instances the DNA encoding the protein of interest is commercially available.

The biologically functional protein can affect cellular mechanism by providing a new or altered function to a cell. For example, the therapeutic polynucleotide can be a multidrug resistance gene (mdr) which encodes P-glycoprotein. P-glycoprotein is a cell membrane glycoprotein which affects intracellular drug accumulation and is responsible for the phenomenon of multidrug resistance.

The therapeutic polynucleotide can encode a non-biologically functional protein. For example, a hybrid gene comprising various domains and functions from a variety of sources can be designed and produced by recombinant technology or enzymatic or chemical synthesis.

The therapeutic polynucleotide may be capable of being transcribed into an RNA molecule which is sufficiently complementary to hybridize to an mRNA or DNA of interest, i.e., a sense or antisense RNA, shRNA, siRNA, miRNA. Such RNA molecules may be useful in preventing or limiting the expression of overproduced, defective, or otherwise undesirable molecules. The AAV vector of the present invention can comprise, as the therapeutic polynucleotide, a sequence encoding an antisense RNA which is sufficiently complementary to a target sequence such that it binds to the target sequence. For example, the target sequence can be part of the mRNA encoding a polypeptide such that it binds to and prevents translation of mRNA encoding the polypeptide. The target sequence may be a segment of a gene that is essential for transcription such that the antisense RNA binds the segment (e.g., a promoter or coding region) and prevents or limits transcription. Hence, the antisense RNA must be of sufficient length and complementarity to prevent translation of its target mRNA or transcription of its target DNA. Antisense RNAs having sufficient complementarity to a target sequence such that the antisense RNA is capable of binding to the target and thereby inhibiting translation or transcription can be determined using standard techniques. The therapeutic polynucleotide can be provided, for example, by chemical or enzymatic synthesis, or from commercial sources.

The function of the AAV vectors of the present invention, i.e., the ability to mediate transfer and expression of therapeutic polynucleotide, can be evaluated by monitoring the expression of the therapeutic polynucleotide in transduced cells. For example, cells may be transduced with an AAV vector of the present invention or infected with varying concentrations of virions containing said AAV vector and then assessed for the expression of the therapeutic polynucleotide.

In some embodiments, a method of producing a rAAV vector drug substance is provided, the method encompassing introducing a helper virus, or a helper virus nucleic acid, or a helper nucleic acid, to a stably transfected mammalian cell line.

### EXAMPLES

Evaluation of Promoters to Regulate shRNAs.

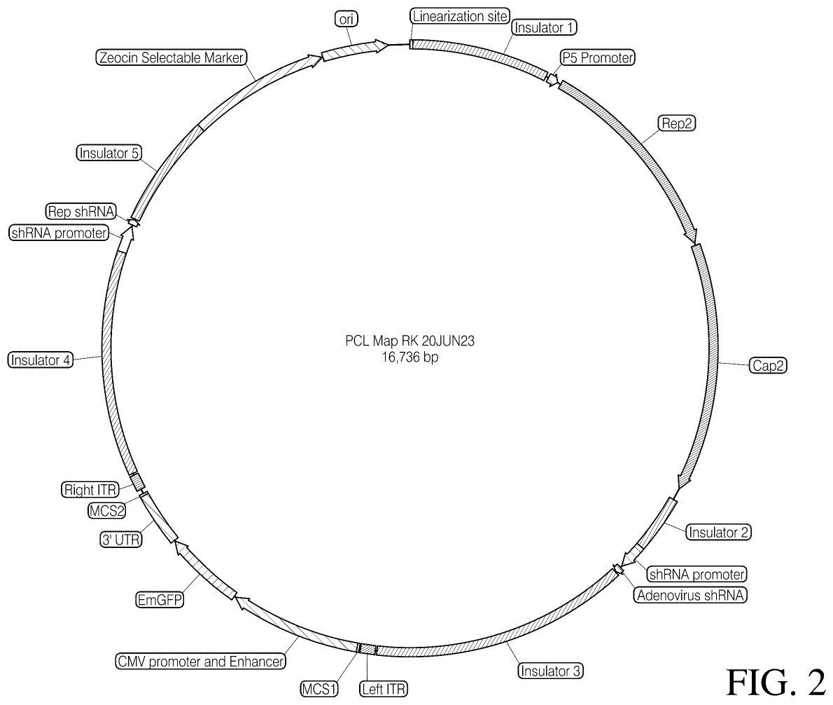

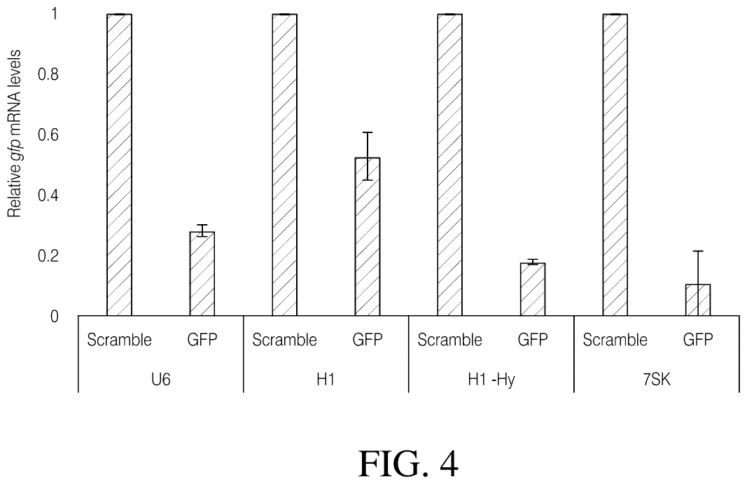

Two distinct shRNA species are included in the disclosed plasmid, for instance, as depicted in FIG. 2, that ultimately provide for the realization of a producer cell line. Given that there are two shRNA sequences, one targeting rep, and another targeting the adenovirus tripartite leader (TPL) sequence, it was necessary to evaluate promoter strength and interactions. To address this, four different promoters were tested. This was accomplished by seeding A549 cells to a confluency of 50-90%. The cells were transfected with a plasmid encoding green fluorescent protein (GFP) under the expression of a PGK promoter and a gfp shRNA sequence under the control of one of four promoters. A scrambled shRNA sequence, a sequence without identity to gfp, was used as a negative control. Transfected cells were harvested 48-72 hours following transfection and gfp mRNA levels were measured by reverse transcriptase polymerase chain reaction (RT-PCR). Results are shown in FIG. 4. All the tested promoters drove sufficient production of shRNA sequence to gfp to reduce gfp levels as determined by RT-PCR.

Evaluation of shRNAs to Regulate Adenovirus Infection.

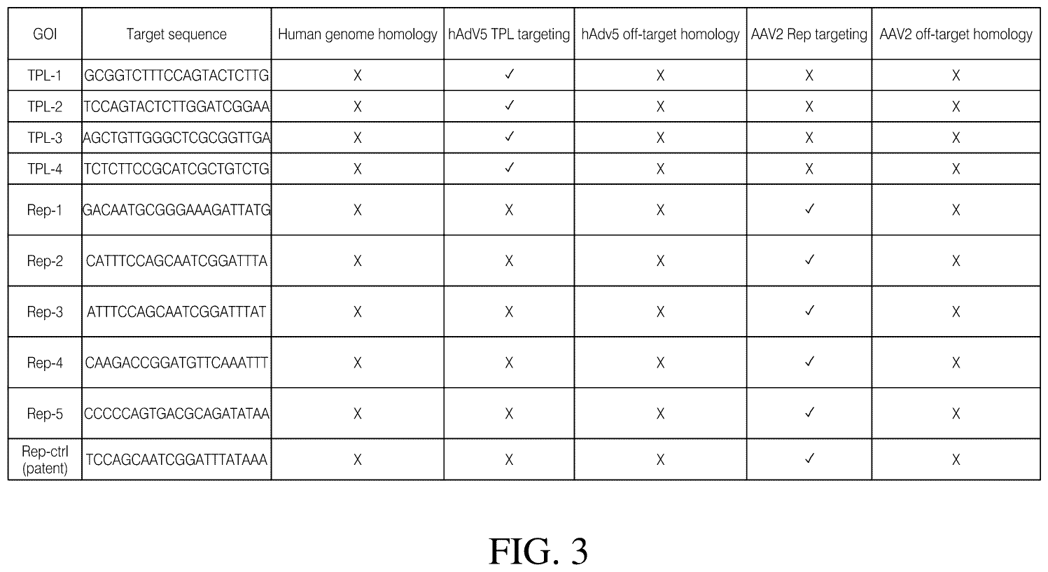

At the outset, it is unpredictable whether a shRNA sequence can be identified which disrupts adenovirus production or infectivity. To address this, a library of shRNA sequences was generated targeting the TPL sequence present on the major late transcription unit of adenovirus type 5 (FIG. 3). A549 cells were seeded and expanded such that a confluency of 50-90% was reached. The cells were transfected with the plasmid encoding the TPL shRNA sequence under the control of the pol III promoter and a Zeocin resistance cassette, using a transfection reagent. A scrambled shRNA sequence, one in which the shRNA nucleotide sequence is randomly arranged, is used as a control sequence. The transfection media may be replaced with fresh medium after 24 hours. Cells are subjected to selection in media containing Zeocin. Surviving colonies were pooled and expanded. The effectiveness of the TPL shRNA was evaluated by seeding the cells such that the confluency is 50-90%. Cells were subsequently infected with wild-type adenovirus type 5 at a duration of 0-48 hours followed by the generation of cell lysates at 48-72 hours post infection. Cells were lysed either by three freeze/thaw cycles or using 0.25%-1% (v/v) Tween 20 and may include an additional endonuclease treatment step. Cellular debris was removed by centrifugation. Cell lysates and/or spent medium were tested for adenovirus titer in the presence of adenovirus.

To determine the optimal time point for adenovirus infection, control experiments are conducted whereby cells are transfected with a plasmid containing the reporter gene, gfp with or without a shRNA sequence targeting gfp. An evaluation of GFP protein expression and knockdown efficiency is measured by fluorescence microscopy or fluorescence-activated cell sorting (FACS). For FACS analysis, cells are detached by addition of TrypLE™ Express Enzyme and resuspended in fresh medium. Other assays for use in determining knockdown efficiency include RT-PCR, for RNA, and Western blot, for protein.

Evaluation of Transcription Components (Promoters and shRNAs) to Regulate Rep Expression.

The purpose of this work is to identify a promoter and shRNA sequence combination which represses Rep expression in the absence of adenovirus type 5 and is rendered ineffective in the presence of adenovirus type 5. The anti-rep shRNA sequences used in this study (4-6 different sequences) are directed against the four mRNAs that encode the AAV Rep proteins, Rep78, Rep68, Rep52 and Rep40. In this study, a suitable shRNA sequence is identified first followed by evaluation of the effectiveness of suppressing Rep expression under the regulation of various promoter types (3-5 promoters). Variation in the promoters is tested for modulation of expression control.

Evaluation of Promoters that Regulate rep shRNA Expression.

To identify a suitable promoter for anti-rep shRNA expression, the anti-rep shRNA that yields knockdown is placed under the control of three to five different promoters in addition to the U6 promoter. Variations for each promoter are included to fine-tune expression further. Adherent A549 and/or adherent HEK293 cells are seeded such that a confluency of ≥50% is reached the next day. Transfection and infection are conducted as described. Measurements of fluorescence, cell viability and REP-GFP mRNA levels occur at 48-72 hrs. post infection.

Evaluation of shRNAs Directed Against Rep Genes.

This study uses a fusion construct of the rep and gfp gene whereby the GFP is fused to the common C-terminus of the four Rep proteins. To confirm that the fusion construct expresses in cells and that the expression can be modulated, adherent A549 and/or adherent HEK293 cells are seeded such that a confluency of 50-90% is reached. The cells are transfected with a plasmid that has the fusion construct (rep-gfp) regulated by the PGK promoter. To confirm that the fusion protein expression can be modulated, the plasmid is designed to include a shRNA sequence targeting gfp (anti-gfp shRNA), whereby the anti-gfp shRNA sequence is under the control of the U6 promoter. A scrambled shRNA sequence is used as a control shRNA sequence. Transfection of the plasmids is conducted using a transfection reagent such as PEI, lipofectamine etc. The transfection media may be replaced with fresh medium after 24 hours. Evaluation of fluorescence of the cells post transfection is conducted as described.

To identify the effectiveness of the anti-rep shRNAs to reduce rep expression, the rep-gfp fusion construct is under the control of the P5 promoter on a plasmid which also has the anti-rep shRNA sequences under the control of the U6 promoter. HEK293 cells express the E1 gene which results in the activation of the P5 promoter thereby driving REP-GFP expression. An evaluation of cell viability and GFP fluorescence uses fluorescence microscopy or FACS. Knockdown of rep uses ELISA or Western blotting and RT-PCR for mRNA detection.

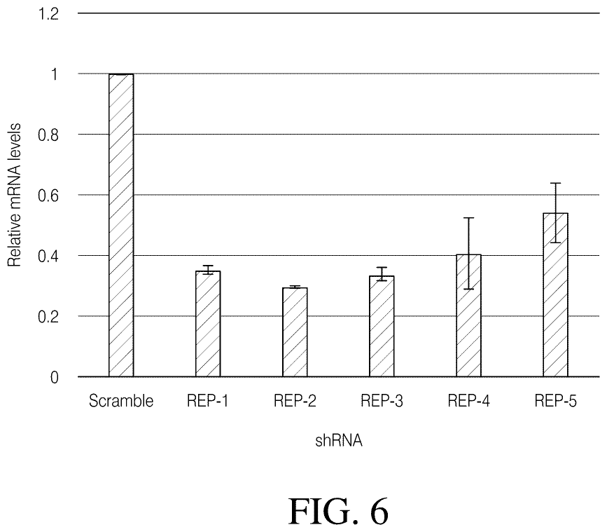

In a series of studies to identify the effectiveness of the anti-rep shRNAs to reduce rep expression, adherent A549 and/or adherent HEK293 cells were seeded such that a confluency of 50-90% was reached. The cells were transfected with a plasmid that has the fusion construct (rep-gfp) under the control of the P5 promoter and the anti-rep shRNA under the control of the U6 promoter. A scrambled shRNA sequence has been used as a control for the anti-rep shRNAs. Cells were transfected with the plasmids using polyethyleneimine (PEI), lipofectamine or another suitable transfection reagent. Evaluation of the rep mRNA levels 24-72 hours post transfection was determined by reverse transcription polymerase chain reaction (RT-PCR) as shown in FIG. 6. As is shown in FIG. 6, expression levels of rep are reduced in the presence of the rep shRNA relative to the scramble sequence.

### Evaluation of Antibiotic Selection for Mammalian Cells

To determine the concentration of zeocin to use for stable transfection, mammalian cells were seeded such that the confluency is >20%. A zeocin kill curve is conducted by subjecting the cells to 50-1000 μg/mL zeocin treatment whereby media changes are conducted 2-4 times a week for a maximum duration of 5 weeks. Cell viability estimates were obtained by visualization under a light microscope. The concentration used for selection for stable transfection is determined by the minimum concentration that kills cells in a target treatment time.

To verify the duration the cells can retain the transfected plasmid for, cells are seeded at a confluency of 50-90% and transfected with a plasmid encoding gene conferring resistance to zeocin under the expression of the promoter identified in eukaryotic and prokaryotic organisms and encoding gfp under a PGK promoter. Cells are placed under zeocin selection at the minimum concentration established above. Surviving cells are pooled, and cultured, and fluorescence is monitored for 1-2 months on a weekly or biweekly basis.

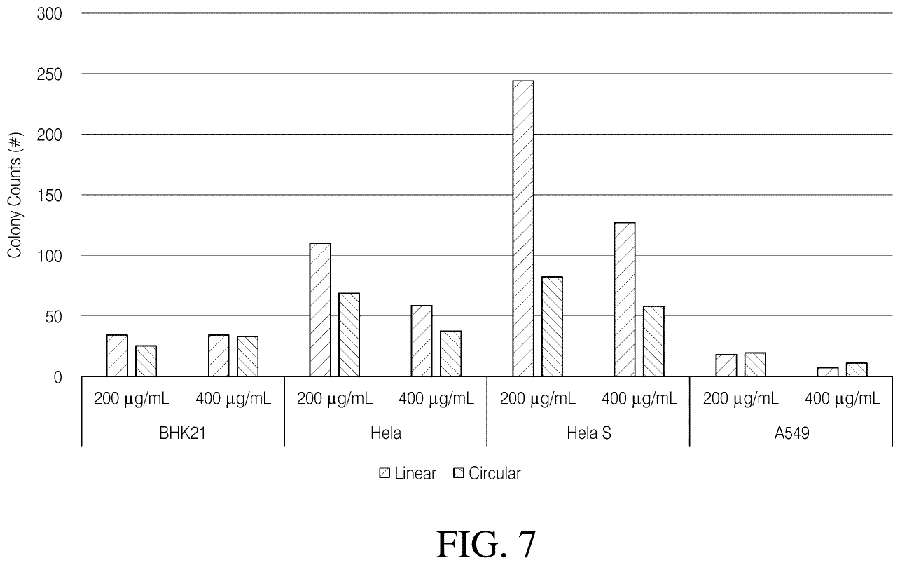

To test whether plasmid linearization impacted retention by transfected cells, cells were seeded to a confluency of 50-90% and transfected with linearized or circular plasmid carrying a zeocin resistance cassette and a gfp gene under the control of the PGK promoter. Transfected cells were placed under zeocin selection. Surviving colonies were counted by visualization under a microscope or by crystal violet staining. Results are shown in FIG. 7. Both linearized and circular plasmids were found to generate stable cells lines. Fluorescence measurements by flow cytometry on pooled colonies confirm the expression of GFP.

To identify the effectiveness of the anti-rep shRNAs to reduce rep expression in stable cells, A549 cells were transfected with a plasmid expressing rep-cap under the control of the P5 promoter, anti-rep shRNA sequences under the control of the U6promoter and a zeocin selection marker. Cells are subjected to selection in media containing zeocin. Surviving colonies are expanded and evaluation of rep mRNA levels was conducted by RT-PCR.

Evaluation of rAAV Production in Various Cells

To evaluate rAAV production, mammalian cells are seeded at a confluency of 50-90%. Cells are transfected with a plasmid encoding gfp between ITRs, resistance gene against zeocin, shRNAs targeting rep and the adenovirus TPL region. The transfection media may be replaced with fresh medium after 24 hours. Cells are infected with adenovirus 24-72 hours post-transfection for 48-72 hrs. Cell lysates are generated. rAAV production is evaluated by vector genome analysis, capsid titer analysis, and infectious titer. Verification of the efficiency of the TPL shRNA is conducted by measuring adenovirus infectious titer.

### Generation of AAV Producer Cell Line

Cells are transfected with the plasmid as described and subjected to selection using zeocin also as described. Single cell clones are isolated and screened for rAAV production and can be frozen.

Clones are thawed and cultured at an increasing scale for ≥3 passages and rAAV production is evaluated by infecting the cells with adenovirus. Top producer clones are evaluated for the stability of rAAV production for ≥15 cell culture passages. Producer cell clones capable of high level rAAV production are master cell banked under GMP and used in the manufacture of AAV vector drug substance that is then formulated into the rAAV drug product.

We claim:

1. A system for producing a recombinant adeno-associated virus (rAAV), the system comprising a stably transfected mammalian cell line and a helper virus, the stably transfected mammalian cell line comprising an exogenous DNA sequence, the exogenous DNA sequence comprising an AAV rep gene, an AAV cap gene, a gene of interest and an RNAi sequence that targets the AAV rep gene expression, the helper virus comprising sequences that increase rAAV vector production upon its introduction into the stably transfected mammalian cell line.

2. The system of claim 1, wherein the exogenous DNA sequence further comprises a selectable marker.

3. The system of claim 1, wherein the exogenous DNA sequence further comprises a second RNAi sequence, wherein the second RNAi sequence targets a helper virus sequence.

4. The system of claim 1, wherein the RNAi sequence comprises a double stranded RNA (dsRNA), a small interfering RNA (siRNA), a small hairpin RNA (shRNA), a micro-RNA or an antisense RNA.

5. The system of claim 4, wherein the RNAi sequence is a shRNA.

6. The system of claim 1, wherein the helper virus is an adenovirus, herpesvirus, Epstein-Bar virus, cytomegalovirus, papillomavirus, bocavirus, or a poxvirus.

7. The system of claim 1, wherein the promoter of the RNAi sequence is an RNA pol II or RNA pol III promoter.

8. The system of claim 7, wherein the promoter of the RNAi sequence is an RNA pol III promoter.

9. The system of claim 3, wherein the promoter of the second RNAi sequence is an RNA pol II or RNA pol III promoter.

10. The system of claim 9, wherein the promoter of the second RNAi sequence is an RNA pol II promoter.

11. The system of claim 9, wherein the promoter of the second RNAi sequence is an RNA pol III promoter.

12. An exogenous nucleic acid comprising an AAV rep gene, an AAV cap gene, an RNAi sequence, wherein the RNAi sequence targets the AAV rep gene expression, a selectable marker, and a gene of interest (GOI) flanked by two AAV inverted terminal repeats (ITRs).

13. The exogenous nucleic acid of claim 12, further comprising a second RNAi sequence, wherein the second RNAi sequence targets a helper virus nucleic acid sequence.

14. The exogenous nucleic acid of claim 12, wherein the rep gene and the AAV cap gene are both from the same AAV serotype.

15. The exogenous nucleic acid of claim 12, wherein the rep gene and the AAV cap gene are both from the different AAV serotypes.

16. The exogenous nucleic acid of claim 12, wherein the AAV rep gene is selected from AAV serotype 1, 2, 3, 4-9, or other known or engineered rep genes.

17. The exogenous nucleic acid of claim 12, wherein the AAV cap gene is selected from AAV serotype 1, 2, 3, 4-9, or other known or engineered cap genes.

18. The exogenous nucleic acid of claim 12, wherein the RNAi is a double stranded RNA, a small interfering RNA, a small hairpin RNA, a micro-RNA or an antisense RNA.

19. The exogenous nucleic acid of claim 18, wherein the RNAi is a small hairpin RNA (shRNA).

20. The exogenous nucleic acid of claim 13, wherein the second RNAi sequence is a double stranded RNA, a small interfering RNA, a small hairpin RNA, a micro-RNA, or an antisense RNA.

21. The exogenous nucleic acid of claim 20, wherein the RNAi is a small hairpin RNA (shRNA).

22. A method for producing a stably transfected cell line, the method comprising transfecting a host cell with the exogenous nucleic acid of claim 12.

23. The method of claim 22, wherein the host cell is a HeLa, HeLa S, BHK, HEK293 or A549 cell.

24. The method of claim 22, wherein the exogenous nucleic acid is targeted to integrate into to a pre-determined site within a host cell genome.

25. A method for producing a recombinant adeno-associated virus (rAAV) vector drug substance, the method comprising introducing a helper virus nucleic acid sequence to a stably transfected mammalian cell line of claim 22 and thereby producing an rAAV drug substance.

26. The method of claim 25, wherein the stably transfected mammalian cell line comprises an exogenous nucleic acid, the exogenous DNA sequence comprising an AAV rep gene, an AAV cap gene, a gene of interest flanked by two AAV inverted tandem repeats, and an RNAi sequence that targets the AAV rep gene expression, the RNAi sequence suppressing AAV rep gene expression.

27. The method of claim 26, wherein the expression of the helper virus helper virus nucleic acid overcomes the suppressive effects of the RNAi sequence that suppresses AAV rep gene expression.

28. The method of claim 26, wherein the exogenous nucleic acid further comprises a second RNAi, the second RNAi targeting a helper virus nucleic acid.

29. The method of claim 28, wherein the second RNAi reduces adenovirus production.

30. The method of claim 25, wherein the helper virus is an adenovirus.
